(19) United States
(12) Patent Application Publication
(10) Pub. No.: US 2025/0144415 A1
(43) Pub. Date: May 8, 2025

(54) NEUROSTIMULATION SYSTEM, APPARATUS, AND METHOD FOR IMPLEMENTING IMPEDANCE CALCULATIONS TO DETERMINE CONDITION OF HYDROGEL PADS

(71) Applicant: AVATION MEDICAL, INC., Columbus, OH (US)

(72) Inventors: Manish Vaishya, Valencia, CA (US); Jacob Ryan Sickafoose, Valencia, CA (US); George Grow, Nashville, TN (US)

(21) Appl. No.: 18/935,913

(22) Filed: Nov. 4, 2024

Related U.S. Application Data
(60) Provisional application No. 63/595,822, filed on Nov. 3, 2023.

(51) Int. Cl. A61N 1/36; A61N 1/04

(52) U.S. Cl. CPC A61N 1/36031; A61N 1/0456; A61N 1/0496

(57) ABSTRACT

A method for determining the condition of hydrogel pads of a transcutaneous neurostimulation system includes providing one or more stimulation electrodes comprising a hydrogel pad through which a transcutaneous neurostimulation signal is applied to skin of the subject. The method also includes providing a controller operatively connected to the stimulation electrodes and being configured to supply a modulated electrical stimulation signal to the stimulation electrodes. The method further includes configuring the controller to measure impedance values of the system and assess changes in system impedance as an indication of the condition of the hydrogel pads.

### RELATED APPLICATIONS

This application claims the benefit of U.S. Provisional Application Ser. No. 63/595,822, filed on Nov. 3, 2023. This application is hereby incorporated by reference in its entirety.

### TECHNICAL FIELD

This disclosure relates to neurostimulation systems, apparatuses, and methods. More specifically, this disclosure relates to neurostimulation systems, apparatuses, and methods that implement impedance calculations for determining the condition of hydrogel pads used to apply transcutaneous electrical stimulation of peripheral nerves.

### BACKGROUND

There are many known technologies that use electrical stimulation of peripheral nerves. Implantable stimulation technologies require surgical implantation of stimulation leads, with a pulse generator that is either surgically implanted or connected externally to wire leads. Percutaneous stimulation technologies are less invasive, but still require the stimulation electrodes to pierce the skin. While these technologies can be effective in treating certain conditions, they are less desirable due to their invasiveness and because they can require the continued or routine attention of specialists, requiring doctor's office visits, phone calls, etc.

### SUMMARY

A neurostimulation system for applying transcutaneous electrical neurostimulation includes an electronic stimulator device or controller that controls the delivery of transcutaneous electrical neurostimulation signal via stimulation electrodes in contact with the subject's skin. The system implements hydrogel pads as an interface between the electrodes and the subject's skin. The controller modulates the electrical neurostimulation signal to apply neurostimulation via the electrodes and hydrogel pads through the skin to the target nerve according to a prescribed treatment regimen.

The neuromodulation system implements an approach to calculating complex impedance values to evaluate both the condition and effectiveness of the hydrogel pad interface between the stimulating electrodes and patient skin. With this data, the system can ensure proper energy is delivered to the target nerve and that the therapy is optimal and comfortable for the patients.

A method for determining the condition of hydrogel pads of a transcutaneous neurostimulation system includes providing one or more stimulation electrodes comprising a hydrogel pad through which a transcutaneous neurostimulation signal is applied to skin of the subject. The method also includes providing a controller operatively connected to the stimulation electrodes and being configured to supply a modulated electrical stimulation signal to the stimulation electrodes. The method further includes configuring the controller to measure impedance values of the system and assess changes in system impedance as an indication of the condition of the hydrogel pads.

According to one aspect, the impedance of the system can include the impedance of the stimulated portions of the human body, the impedance of the hydrogel pads, and the impedance of the skin-hydrogel pad interface.

According to another aspect, alone or in combination with other aspects, configuring the controller to measure impedance values of the system can include configuring the controller to implement a simplified electrical model for the biological impedance of the neurostimulation system.

According to another aspect, alone or in combination with other aspects, the modulated electrical stimulation signal can be a modulated constant current square wave electrical stimulation signal.

According to another aspect, alone or in combination with other aspects, the method can also include measuring the voltage of the stimulation signal applied via the constant current square wave electrical stimulation signal to estimate the simplified impedance via the simplified model at any given point in the wave.

According to another aspect, alone or in combination with other aspects, measuring the voltage of the stimulation signal applied via the constant current square wave can include calculating the impedance as a function of frequency.

According to another aspect, alone or in combination with other aspects, calculating the impedance as a function of frequency can include implementing a Fourier transform.

According to another aspect, alone or in combination with other aspects, configuring the controller to assess changes in impedance of the system can include implementing one or more lookup tables that correlate hydrogel pad condition with system impedance.

According to another aspect, alone or in combination with other aspects, implementing one or more lookup tables can include that correlate hydrogel pad condition with static system impedance determined as a function of frequency.

According to another aspect an apparatus for applying electrical stimulation to a target peripheral nerve in a subject can include one or more stimulation electrodes comprising a hydrogel pad, a wearable structure for supporting the stimulation electrodes, and a controller for controlling the operation of the stimulation electrodes. The control unit can be configured to energize the stimulation electrodes to apply stimulation to a target nerve according to the method according to any of the previous aspects. A neurostimulation system can include this apparatus.

### DRAWINGS

FIG. 1 is a schematic illustration of a neurostimulation system, according to an example configuration.

FIG. 2 is a common human body electrical model.

FIG. 3 is a simplified model circuit implemented in the neurostimulation system.

FIG. 4 is a chart illustrating the voltage response to a constant current signal applied to the model circuit of FIG. 3.

FIG. 5 is a chart illustrating the varying voltage output across the human body when a constant current square wave is applied.

FIG. 6 is a chart illustrating the impedance separation across the frequency band for a neurostimulation system that includes electrodes with and without hydrogel pads.

### DESCRIPTION

FIG. 1 illustrates an example system 10 for delivering transcutaneous neurostimulation to a subject. The neurostimulation system 10 includes an apparatus in the form of a controller 12 (e.g., a microcontroller) and one or more electrodes 14 (E₁, E₂, E₃, . . . Eₙ) configured to be positioned on a skin surface. The positioning of the electrodes 14 on the skin surface can be achieved in a variety of manners. For example, the electrodes 14 can be manually positioned on the skin surface, such as by placing stick-on disposable electrodes directly on the skin. In other implementations, the system 10 can include a wearable 16, such as a strap, brace, sock, sleeve, wrap, or garment, upon which the electrodes 14 and/or the controller 12 can be mounted. In this configuration, the electrodes can be positioned in contact with the skin surface when the wearable 16 is placed on the subject.

As shown in the detail of FIG. 1, regardless of the implementation, a hydrogel pad 22 is positioned between the electrode 14 and the patient's skin. The hydrogel pad 22 provides the electrical interface between the electrode 14 and the skin. The hydrogel pad 22 conforms to the skin and provides a reliable physical contact with the patient through which the electrical connection can be established.

The system 10 is configured to apply electrical stimulation signals to one or more nerves of the subject through the skin according to a prescribed neurostimulation method. While these methods can vary widely, they all entail varying or modulating the applied electrical neurostimulation signal, a process referred to herein as neuromodulation. To do so, a neurostimulation circuit 20 embedded in the controller 12 includes a constant current source that is controlled to produce the neuromodulation signal. The neurostimulation circuit 20 is capable of high voltage biphasic output applied across a load which, in the neurostimulation setting, includes the stimulated tissue.

The neurostimulation system 10 can be implemented in a wearable, such as a garment, sock, sleeve, brace, strap, etc. The wearable includes the components of the apparatus, e.g., the controller 12, electrodes 14, and hydrogel pads 22. Advantageously, the wearable allows the subject to use the system at a time and place that is convenient. The subject may choose to use the device while they are at work or at home, or while walking, relaxing, or sleeping, as long as certain environments and/or activities (e.g., wet environments/activities) are avoided. Since there are no implantable or percutaneous components, the risk of infection, battery fault burns, and transcutaneous power transfer discomfort and/or bleeding, are greatly reduced or eliminated.

The wearable includes the electrodes 14/hydrogel pads 22 arranged in a predetermined pattern or array, and that engage the subject's skin at desired locations when the wearable is worn. The electrodes can include stimulating electrodes and recording electrodes, which the wearable can position at the same location or at different locations on the subject's skin. In fact, the identities of individual electrodes, i.e., stimulating or recording, can change depending on the application/treatment for which the system is being used. The stimulating electrodes apply the transcutaneous electrical stimulation to the subject's skin, and the recording electrodes record the electromyogram (EMG) responses elicited by the stimulation.

The controller 12 is electrically connected to the electrodes 14 and is operable to control electrical stimulation applied by the stimulating electrodes and to control the recording of EMG responses by the recording electrodes. The controller 12 can execute closed-loop control algorithms, which adjust stimulation patterns, periodically or constantly, based on the elicited EMG response from the recruited nerves as feedback. Alternatively, the system 10 can implement open-loop control where stimulation is applied without feedback.

Closed-loop control can eliminate the need for programming sessions commonly required for neurostimulation systems. The day-to-day variability that arises due to electrode placement and skin impedance necessitates these sessions to make sure that the electrodes are positioned to provide adequate stimulation treatment. With the present system, instead of physically adjusting the electrode positions on the subject in order to find the arrangement that produces the desired response, the system itself can select which electrodes to use, and can adjust the number and pattern of electrodes until an acceptable response (EMG and/or MMG) is achieved.

Once the appropriate electrodes pattern is identified, the order, intensity, timing, etc. of the stimulation can be further tuned or adjusted to optimize the EMG and/or MMG response. The system can tailor the electrical stimulation applied by each individually controllable electrode in the array so that the stimulation characteristics of each electrode (e.g., frequency, amplitude, pattern, duration, etc.) is configured to deliver the desired stimulation effect. This tailoring can be implemented automatically through the algorithm, which incrementally adjusts these characteristics, monitoring the and/or response at each increment until optimal settings are identified. Stimulation therapy can then be applied with these settings, according to the algorithm, which can be dictated by the requirements of the treating physician.

Alternatively, a fixed number of electrodes arranged in a fixed pattern can be implemented. In this implementation, the stimulation parameters (e.g., frequency, amplitude, pattern, duration, etc.) can be tailored or adjusted systematically to determine the initial stimulation settings for closed-loop or open-loop stimulation. Again, this tailoring can be implemented automatically through the algorithm, which incrementally adjusts these characteristics, monitoring the and/or response at each increment until optimal settings are identified. Stimulation therapy can then be applied with these settings, according to the algorithm, which can be dictated by the requirements of the treating physician.

The control unit and the architecture of the system may be designed to constantly optimize stimulation by monitoring the quality of nerve recruitment periodically or on a pulse-by-pulse basis, with the goal of keeping recruitment strength to a minimum (which can reduce muscle twitching) and to minimize the stimulation energy being delivered through the skin. The EMG recording feature is capable of detecting both M-wave and F-wave responses, which can be used as feedback inputs (together or independently) to the closed-loop stimulation algorithm to determine the level of activation of the stimulated peripheral nerve. A significant aspect of the F-wave is that it provides an indication that the stimulation-evoked peripheral nerve action potential has activated motor neurons in the associated spinal cord nerves/nerve plexus. For example, an F-wave response to tibial nerve stimulation indicates that the tibial nerve action potential has activated motor neurons in the sacral spinal cord/sacral plexus.

The wearable transcutaneous electrical stimulation device can be used to stimulate various peripheral nerves in order to treat medical conditions associated with those nerves. For example, the system can be used to apply electrical stimulation to the tibial nerve to treat pelvic floor dysfunction, e.g., overactive bladder (OAB) medical conditions. As another example, the system can be used to apply electrical stimulation to the tibial nerve to treat sexual dysfunction. In this manner, it is believed that tibial nerve stimulation could be used to treat genital arousal aspects of female sexual interest/arousal disorder by improving pelvic blood flow. In yet another example, the system can be used to apply electrical stimulation to the tibial nerve to treat plantar fasciitis.

As another example, the system can be applied to the wrist area to provide stimulation to the ulnar nerve and/or median nerve. The stimulation electrode array can, for example, be placed on the inside of the lower arm anywhere 0 to 20 cm from the wrist line. EMG recording electrodes can be placed on the base of thumb to record signal from abductor/flexor pollicis brevis. EMG recording electrodes alternatively or additionally can be placed on the base of pinky to record signal from abductor/flexor digiti minimi brevis. The nerve activation could be confirmed by recording M-wave and F-wave EMG signals from the relevant muscles. The EMG signal can also be used as a control signal to adjust the stimulation parameters or stimulation electrode patterns. This technology can be applied to median nerve activation for pain management in carpal tunnel syndrome, hypertension management, and nerve conduction study/nerve injury diagnosis for median/ulnar nerve neuropathy, etc.

As a further example, the system can be used to apply transcutaneous electrical stimulation to provide neurostimulation to peripheral nerves in order to enhance nerve regeneration after peripheral nerve injury.

Implementing closed-loop control, the system can utilize measured EMG responses to detect and obtain data related to the electrical activity of muscles in response to the applied stimulation. This data can be used as feedback to tailor the application of the electrical stimulation. Additionally or alternatively, the system can also implement MMG sensors, such as accelerometers, to measure the physical response of the muscles. Other feedback, such as impedance measurements between electrodes and other biopotential recording, can also be utilized. Through this closed-loop implementation, the system can utilize techniques such as current steering and nerve localization to provide peripheral nerve stimulation therapy for treating various medical conditions.

### Determining Hydrogel Pad Condition

One factor that can affect the performance of the neurostimulation system 10 is the condition of the hydrogel pads 22. The system 10 is configured to implement and evaluate a simplified human electrical model to help determine the condition of the hydrogel pads 22 in real-time during while neurostimulation is being delivered to the subject.

Development of an electrical model to capture the impedance response of the human body has been done by researchers in the past. Some of the research states that electrical impedance within the tissue tends to be linear but the impedance of the skin/electrode interface is frequency dependent. This means that there is both a resistive and reactive element to the impedance through the human body. Many of these research papers use a complex network of resistors and capacitors to model the human body. Despite this, even the most complex network may be simplified mathematically, without significantly affecting the accuracy of the model.

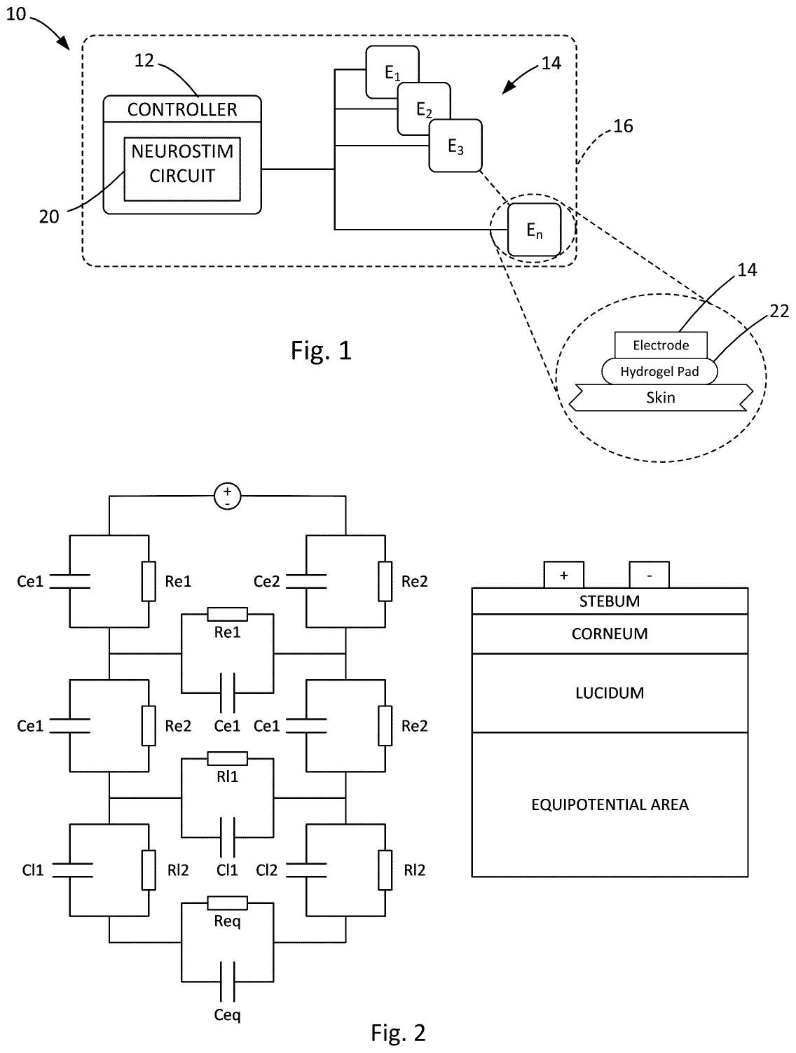

Development of an electrical model to capture the impedance response of the human body has been the topic research that states that the electrical impedance of the skin/electrode interface is frequency dependent while the impedance within the tissue tends to be linear. This means that there is both a resistive and reactive element to the impedance of the human body. These research papers typically use a complex network of resistors and capacitors to most accurately model the human body. An example of this is shown in FIG. 2, which shows an example of a human model circuit next to a schematic representation of the electrode interfacing the epidermis. FIG. 2 also illustrates all the connections between the electrodes siphoning current away from the targeted nerve.

The neurostimulation system 10, particularly the controller 12, implements a simplified practical model to estimate the overall system impedance that includes the human body, the hydrogel pads and the skin-to-hydrogel interface, and, using further statistical analysis, predicts the presence or the condition of the hydrogel pads that are used between the skin and stimulation electrodes. The system 10 outputs a constant current square wave for neuromodulation and, utilizing this the voltage response from anode to cathode across the human body, the hydrogel pads and the skin-to-hydrogel interface can be measured to derive the impedance as a function of time. Using the time variable impedance, the simplified resistive and capacitive values can be calculated in order to determine the full complex impedance of the circuit. With the full system impedance calculated, the condition and effectiveness of the hydrogel pads can be empirically determined by analyzing the variability in impedance value and cataloging the impedance as a function of varying conditions of the hydrogels, such as dryness, physical condition or number of cycles of use.

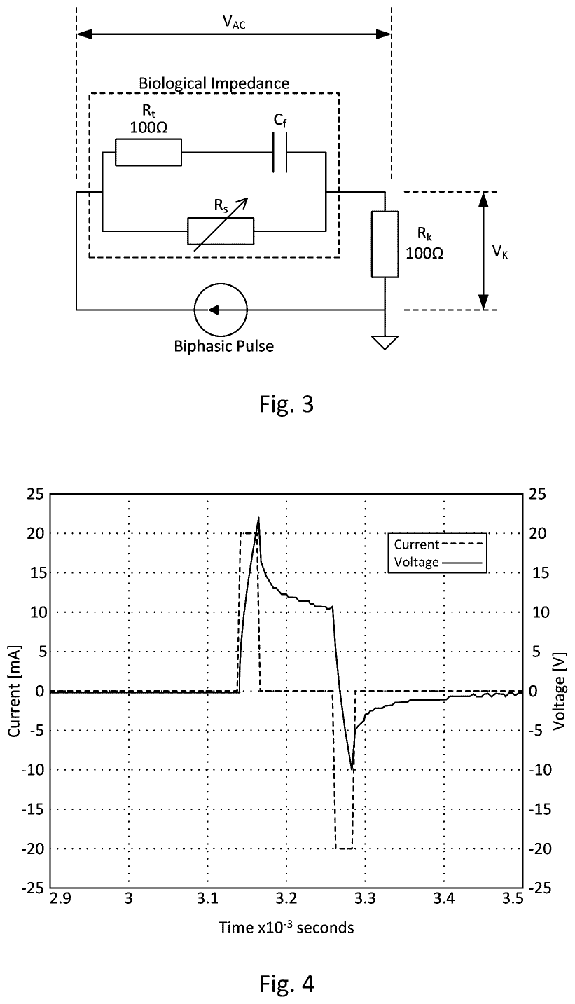

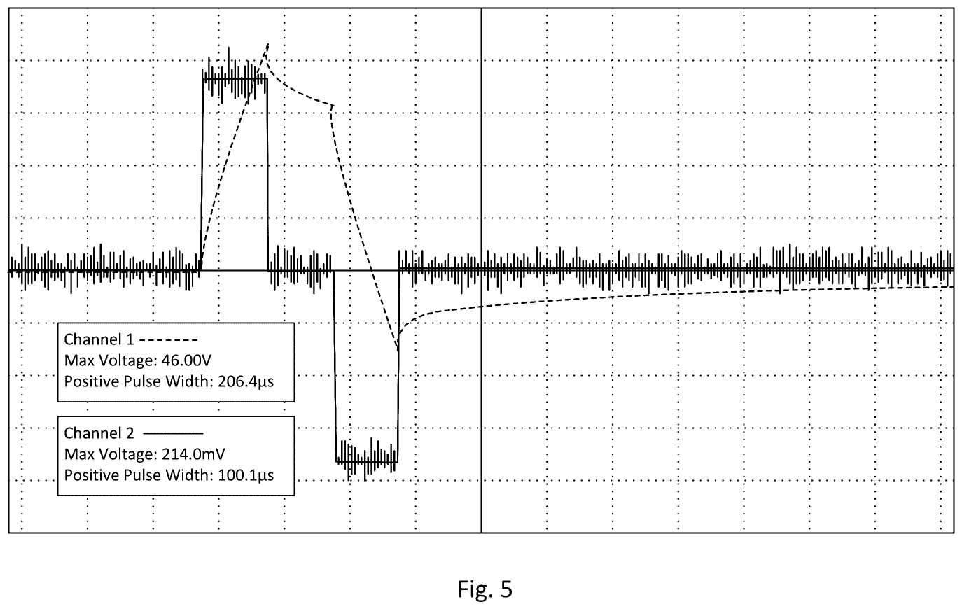

To accomplish this, the system 10 implements a simplified resistor/capacitor network of the human model of FIG. 2. This simplified resistor/capacitor network is shown in the representative circuit of FIG. 3. As shown in FIG. 3, the biological complex impedance is modeled with both resistance and capacitance. The biphasic constant current pulse of the stimulation current applied across the biological impedance and resistive load Rₖ. The voltage response V_{K} to a constant current signal sent through this model circuit is shown in FIG. 4.

Because the system 10 outputs a constant current square wave, the voltage V_{K} can be measured to estimate the impedance at any given time point i. This is done using the simple Ohm's law equation, extended to AC signals, to calculate complex impedance:

$$Z = \frac{{V}_{K}}{I}$$

FIG. 4 shows the varying voltage output (solid line) across the human body when a constant current square wave (dashed line) is applied.

Since the overall system impedance is constituted of human body impedance, hydrogel impedance, and that of the interface between the skin and the hydrogel, a damaged, dried or missing hydrogel pad will cause the hydrogel and interface impedance to increase. First, a database is created to correlate the condition of the hydrogel with the static impedance value as well as impedance resulting from the absence of the hydrogel interface. Employing lookup tables, and by measurement of the impedance value during use by patients, the condition of the hydrogels can be interpolated, and further refined using known statistical methods.

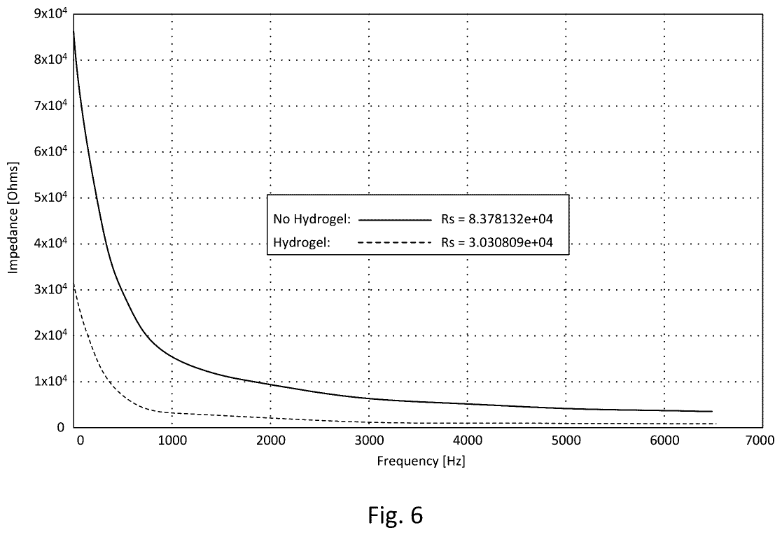

Going beyond the simple equation to find impedance at a given point, the system 10 is configured to implement a Fourier transform to analyze the square wave calculate the impedance magnitude as a function of frequency. By breaking down the signal into frequency bands, the difference in impedance is compared to static measurements, given the complex characteristics of the hydrogel electrical properties, including capacitance, inductance and resistance. Thus, graphing impedance magnitude as a function of frequency allows the system 10 to further magnify the effect of hydrogel condition, and both detect the presence of the hydrogel pads 22 and to calculate/determine their condition. FIG. 6 shows the impedance separation across the frequency band with and without the hydrogel pads 22 between the skin and the electrodes 14. As shown in FIG. 6, the impedance separation is more distinct at lower frequency bands.

From the above, it will be appreciated that the system 10 is configured to utilize impedance calculations to determine the condition of hydrogel pads 22. To do this, the system 10 implements a simplified electrical model for the impedance of the entire system 10, including the stimulated portions of the human body, using the constant current signal from active neuromodulation. A frequency-domain analysis can further magnify the results, by observing the impedance in different frequency bands.

Various examples of the disclosure have been described. These and other examples are within the scope of the following claims.

We claim:

1. A method for determining the condition of hydrogel pads of a transcutaneous neurostimulation system, comprising:
providing one or more stimulation electrodes comprising a hydrogel pad through which a transcutaneous neurostimulation signal is applied to skin of the subject;
providing a controller operatively connected to the stimulation electrodes and being configured to supply a modulated electrical stimulation signal to the stimulation electrodes; and
configuring the controller to measure impedance values of the system and assess changes in system impedance as an indication of the condition of the hydrogel pads.

2. The method recited in claim 1, wherein the impedance of the system comprises the impedance of the stimulated portions of the human body, the impedance of the hydrogel pads, and the impedance of the skin-hydrogel pad interface.

3. The method recited in claim 2, wherein configuring the controller to measure impedance values of the system comprises configuring the controller to implement a simplified electrical model for the biological impedance of the neurostimulation system.

4. The method recited in claim 2, wherein the modulated electrical stimulation signal is a modulated constant current square wave electrical stimulation signal.

5. The method recited in claim 2, wherein the method further comprises measuring the voltage of the stimulation signal applied via the constant current square wave electrical stimulation signal to estimate the simplified impedance via the simplified model at any given point in the wave.

6. The method recited in claim 5, wherein measuring the voltage of the stimulation signal applied via the constant current square wave comprises calculating the impedance as a function of frequency.

7. The method recited in claim 5, wherein calculating the impedance as a function of frequency comprises implementing a Fourier transform.

8. The method recited in claim 1, wherein configuring the controller to assess changes in impedance of the system comprises implementing one or more lookup tables that correlate hydrogel pad condition with system impedance.

9. The method recited in claim 8, wherein implementing one or more lookup tables comprises that correlate hydrogel pad condition with static system impedance determined as a function of frequency.

10. An apparatus for applying electrical stimulation to a target peripheral nerve in a subject according to claim 1, comprising:
one or more stimulation electrodes comprising a hydrogel pad;
a wearable structure for supporting the stimulation electrodes; and
a controller for controlling the operation of the stimulation electrodes and for implementing the method of claim 1, wherein the control unit is further configured to:
energize the stimulation electrodes to apply stimulation to a target nerve.

11. A neurostimulation system comprising the apparatus of claim 10.
